(19) United States
(12) Patent Application Publication
(10) Pub. No.: US 2007/0280879 A1
(43) Pub. Date: Dec. 6, 2007

(54) Therapeutic Use Of Rm1 Antigen

(75) Inventors: Mark Glassy, San Diego, CA (US); Michael E. McKnight, Poway, CA (US); Sonjoy Mukerjee, Hyderabad (IN); Subhra Chakrabarti, Hyderabad (IN)

Correspondence Address: WILSON SONSINI GOODRICH & ROSATI, 650 PAGE MILL ROAD, PALO ALTO, CA, 94304-1050, US

(73) Assignee: Shantha West, Inc., San Diego, CA (US)

(21) Appl. No.: 10/592,401

(22) PCT Filed: Mar. 11, 2005
(86) PCT No.: PCT/US05/08264

§ 371 Date: Aug. 3, 2007

Related U.S. Application Data
(60) Provisional application No. 60/552,656, filed on Mar. 11, 2004.

(51) Int. Cl. A61K 38/16; A61K 36/00; A61K 39/395; A61K 49/00; A61K 51/00; A61P 35/00; A61P 37/00; A61P 43/00; C07H 21/00; C07K 14/00; C12N 1/00; C12N 1/15; C12N 15/63; C12N 5/00; G01N 33/53

(52) U.S. Cl. 424/1.11; 424/130.1; 424/725; 424/9.2; 435/243; 435/254.11; 435/320.1; 435/325; 435/7.2; 514/12; 530/358; 536/23.5

(57) ABSTRACT

The antigen recognized by RM1 antibody is used for therapy, diagnosis and imaging.

### BACKGROUND OF THE INVENTION

Novel protein expression patterns are associated with abnormal cell growth, especially cancer. One type of over-expressed proteins are known as tumor associated antigens (TAAs).

Some of these TAAs are expressed only infrequently by an individual tumor. Other TAAs may be shared between histologically similar tumors, while others may be expressed by a variety of histologically distinct cancers as well as fetal tissues.

TAAs of the former kind are of little use except in devising an autologous treatment method. In contrast, TAAs that are shared between a wide range of tumors that are histologically similar or even histologically dissimilar have potential applications for immunodiagnosis, immunoprognosis and treatment of patients across a wide number of malignancies.

An example of the first type of TAA is the idiotype protein of a B cell lymphoma. One example of the more widespread TAAs is the MAGE protein isolated from melanoma cells.

Another type of over-expressed or aberrantly expressed proteins associated with tumors are the expression products of oncogenes. Inhibiting the activity of oncogenes has proven to be a useful means of therapy.

There are examples which suggest that raising immunity against growing neoplasm in humans can be enhanced by active immunization with TAAs or the expression product of oncogenes. The purpose of such active specific immunization is induce an immune response to antigens associated with the growth or metastasis of tumors beyond that which is naturally induced by the growing neoplasm. It is believed that a growing neoplasm does not induce a maximum immune response in the host to the proteins that are expressed at increased or aberrant levels.

Immunization with TAAs are proving effective in the clinic. Animal models have also demonstrated the efficacy of raising an immune response to oncogenes associated with transformation.

Therefore, if a means can be provided to enhance the response to antigens associated with these abnormally expressed proteins, it would prove useful in cancer therapy. Further, identification of specific antigens could also prove useful in the diagnosis, imaging, and staging of cancers.

In particular, lung cancer is the most common lethal cancer in the United States. In 1992, 168,000 new cases and 146,000 deaths from lung cancer were estimated. The overall five-year survival rate for newly diagnosed cases of non-small cell lung cancer (NSCLC) is only 10-15%. Therefore, novel means of treating lung cancer would prove very useful, as well as means of detecting or imaging lung cancer.

Colon cancer is the second most frequently diagnosed malignancy in the U.S. Although it is often curable by resection if confined to the bowel, colon cancer is nevertheless the second leading cause of cancer death in the U.S. Therefore, novel means of treating, diagnosing, or imaging colon cancer would prove useful.

### SUMMARY OF THE INVENTION

In one aspect, the instant invention features an isolated histone H2A polypeptide that specifically binds to a human monoclonal antibody produced by the cell line deposited as ATCC accession no. CRL-12142. This antibody and cell line have been previously described in U.S. Pat. No. 5,744,585 (named inventors: Rajko D. Medenica and Sonjoy Mukerjee) which is hereby incorporated in its entirety. In one embodiment of this aspect, this invention features a H2A histone that has a deletion at the amino terminus relative to full length native human histone H2A. In a certain, but not limiting, embodiment the isolated histone H2A polypeptide of the invention begins with the amino terminal sequence AAVLGYLTAEILELA. In another non-limiting embodiment, the isolated histone H2A polypeptide of the invention begins with the amino terminal sequence MAAVLEYLTAEILELA, and in a slightly different, non-limiting embodiment, it begins with AAVLEYLTAEILELA.

In specific embodiments of this aspect, this invention features a H2A histone that has a deletion of at least 10 amino acids from the amino terminus, relative to full length native human histone H2A; at least 20 amino acids from the amino terminus, relative to full length native human histone H2A; at least 30 amino acids from the amino terminus, relative to full length native human histone H2A; at least 40 amino acids from the amino terminus, relative to full length native human histone H2A; at least 50 amino acids from the amino terminus, relative to full length native human histone H2A; 51 amino acids from the amino terminus, relative to full length native human histone H2A; at least 60 amino acids from the amino terminus, relative to full length native human histone H2A; at least 70 amino acids from the amino terminus, relative to full length native human histone H2A; or at least 80 amino acids from the amino terminus, relative to full length native human histone H2A.

In further embodiments of this aspect, this invention features a H2A histone that has a deletion of between 40 and 60 amino acids from the amino terminus, relative to full length native human histone H2A; or of between 45 and 55 amino acids from the amino terminus, relative to full length native human histone H2A.

In still further embodiments of this aspect, this invention features a H2A polypeptide no more than 128 amino acids in length; a H2A polypeptide no more than about 90 to about 100 amino acids in length; a polypeptide no more than about 70 to about 90 amino acids in length; or a polypeptide no more than about 50 to about 70 amino acids in length.

In other aspects, the instant invention features an isolated polypeptide including a carboxy-terminal portion of an H2A polypeptide that specifically binds to a human monoclonal antibody produced by the cell line deposited as ATCC accession no. CRL-12142, where this H2A sequence is no more than 128 amino acids in length; where this H2A sequence is no more than about 100 amino acids in length; where this H2A sequence is no more than about 90 amino acids in length; where this H2A sequence is no more than about 80 amino acids in length; where this H2A sequence is no more than about 70 amino acids in length; where this H2A sequence is no more than about 60 amino acids in length; where this H2A sequence is no more than about 52 amino acids in length; or where this H2A sequence is no more than about 50 amino acids in length.

In some embodiments of the above aspects, the H2A polypeptide sequence is mammalian. In other embodiments of the above aspects, the H2A polypeptide sequence is human.

In further embodiments of the above aspects, the H2A polypeptide sequence is as set forth in FIG. 1.

In still further embodiments of the above aspects, the H2A polypeptide sequence is an H2A variant. This H2A variant may be selected from the group represented by GenBank Accession Nos. AF058445; AF058446; AF044286; AF058444; NM138609; NM138609; BC013331; AF054174; AF041483; NM138610.1; and NM004893.2. The histone variant may also be selected from a member of the group set forth in Table 1 as accession numbers for their amino acid sequences.

TABLE 1

| | | |
|---|---|---|
| AAH13331.1 | AAH66236.1 | AAA63191.1 |
| NP_613075.1 | AAH66235.1 | AAA37809.1 |
| AAC33434.1 | NP_002096.1 | AAN59967.1 |
| AAH60324.1 | AAH11694.1 | NP_066409.1 |
| AAT68255.1 | AAH13416.1 | CAB39197.1 |
| NP_003508.1 | AAH04915.1 | P20671 |
| CAI_12569.1 | P16104 | CAB02538.1 |
| Q16777 | CAA32968.1 | CAG46796.1 |
| AAN59959.1 | NP_066408.1 | CAG46768.1 |
| AAH19308.1 | NP_003505.1 | AAN59966.1 |
| AAH01629.1 | NP_003502.1 | AAH85010.1 |
| NP_003507.1 | NP_003501.1 | AAH17379.1 |
| CAI12565.1 | NP_003500.1 | NP_003503.1 |
| CAI12562.1 | AAN59974.1 | AAH50602.1 |
| AAC24465.1 | AAN59973.1 | AAB82086.1 |
| CAG46670.1 | AAN59972.1 | AAB53429.1 |
| CAG38762.1 | AAN59970.1 | CAB02540.1 |
| P20670 | AAN59968.1 | Q93077 |
| AAN59957.1 | AAH71668.1 | AAN59965.1 |
| AAN08620.1 | AAH32756.1 | NP_734466.1 |
| NP_613258.1 | CAA16948.1 | AAH62211.1 |
| NP_004884.1 | CAA15669.1 | CAC44614.1 |
| BAB14565.1 | CAD24077.1 | AAN59963.1 |
| AAC33433.1 | CAD24073.1 | AAH01193.1 |
| NP_542163.1 | CAB11417.1 | AAH82269.1 |
| AAN59969.1 | AAH69306.1 | NP_835736.1 |
| CAA16944.1 | AAH16677.1 | NP_254280.1 |
| NP_060737.1 | CAB06037.1 | CAI23331.1 |
| BAA91894.1 | CAB06034.1 | AAN59960.1 |
| NP_808760.1 | HSHUA1 | NP_778235.1 |
| AAH03602.1 | AAC24466.1 | CAI12570.1 |
| NP_066544.1 | CAA58539.1 | AAN59958.1 |
| AAN59971.1 | CAA40417.1 | P04908 |
| CAB81656.1 | P02261 | CAA24951.1 |
| CAB06031.1 | NP_003504.2 | HSHUA5 |
| Q99878 | NP_066390.1 | CAG33360.1 |
| AAH66234.1 | AAN59964.1 | |
| AAH66232.1 | CAB39192.1 | |
| AAH66233.1 | CAB06036.1 | |
| AAH66237.1 | G40335 | |

In another aspect, the instant invention features a nucleic acid encoding any individual one of the above polypeptides. This nucleic acid may comprise additional nucleic acid sequences, or may consist essentially of a nucleic acid sequence encoding any one of the polypeptides described herein. In a related aspect, the invention features a vector comprising a nucleic acid encoding a H2A histone polypeptide of the invention.

In yet another aspect, the instant invention features a transformed cell that contains any one of the polypeptides described herein. Also, in a related aspect, the instant invention features a transformed cell that contains a nucleic acid encoding any of the H2A histone polypeptides of the invention. In embodiments of the invention, the transformed cell is a prokaryotic cell or a eukaryotic cell. In some embodiments of the invention, the cell is a bacteria, a fungal cell, an insect cell, or a mammalian cell.

In an aspect, the instant invention features a pharmaceutical composition comprising any H2A polypeptide of the invention, and a pharmaceutically acceptable carrier. In a further aspect, the instant invention features a kit comprising any H2A polypeptide of the invention or a pharmaceutical composition comprising any H2A polypeptide of the invention.

In an additional aspect, the instant invention features a method of identifying a cell that expresses a histone H2A polypeptide of the invention that specifically binds to a human monoclonal antibody produced by the cell line deposited as ATCC accession no. CRL-12142, comprising screening the cell for expression of a histone H2A polypeptide of the invention that specifically binds to a human monoclonal antibody produced by the cell line deposited as ATCC accession no. CRL-12142. In an embodiment of this aspect, the instant invention features a method of identifying a cell that expresses a histone H2A polypeptide of the invention on the cell surface that specifically binds to a human monoclonal antibody produced by the cell line deposited as ATCC accession no. CRL-12142, comprising screening the cell for expression of a histone H2A polypeptide that specifically binds to a human monoclonal antibody produced by the cell line deposited as ATCC accession no. CRL-12142.

In another embodiment of this aspect, the screening includes detecting the presence of the histone H2A polypeptide of the invention. In another embodiment of this aspect, the screening includes detecting the presence of a nucleic acid sequence encoding amino acid sequence of the histone H2A polypeptide of the invention.

In an embodiment of this aspect, the cell is present in a subject or a patient. The subject or patient may be a mammal; the subject or patient may be a human.

In a further aspect, the invention features a method of screening for the presence of a cell proliferative disorder, where this method includes analyzing a biological sample for the presence of a histone H2A polypeptide of the invention that specifically binds to a human monoclonal antibody produced by the cell line deposited with the ATCC under accession no. CRL-12142.

The method of this aspect may include the step of detecting the presence of a histone H2A polypeptide of the invention, or it may include the step of detecting the the presence of a nucleic acid encoding a histone H2A polypeptide of the invention. In this aspect, the presence of the invention histone H2A polypeptide or a nucleic acid encoding the amino acid sequence of the histone H2A polypeptide of the invention may be detected in vivo or in vitro.

In embodiments of aspects of the invention, the cell proliferative disorder being detected is, in whole or in part, a benign hyperplasia or a tumor. The tumor being detected may be metastatic; the tumor may be classified as a stage I, II, III, IV or V tumor; the tumor may be a solid tumor or a liquid tumor. The tumor being detected may be hematopoetic. The tumor being detected may be a sarcoma, carcinoma, melanoma, myeloma, blastoma, glioma, lymphoma or leukemia. The cell proliferative disorder being detected may comprise a cell selected from or arising from a breast, colon, gut, lung, brain, skin or pancreas cell.

In an additional aspect, the instant invention includes a method of inducing or increasing an immune response to a histone H2A polypeptide that specifically binds to a human monoclonal antibody produced by the cell line deposited as ATCC accession no. CRL-12142, where this method includes administering to a subject or patient a therapeutically effective amount of a histone H2A polypeptide of the invention that specifically binds to a human monoclonal antibody produced by the cell line deposited as ATCC accession no. CRL-12142 to elicit an immune response to the histone H2A polypeptide in the subject or patient. Such a therapeutically effective amount of a histone H2A polypeptide of the invention that specifically binds to a human monoclonal antibody produced by the cell line deposited as ATCC accession no. CRL-12142 to elicit an immune response to the histone H2A polypeptide in the subject may be referred to as an immunogen comprising the histone H2A polypeptide of the invention. The induced immune response may comprise a cell-mediated or a humoral immune response.

In a further aspect, the invention features a method of treating a cell proliferative disorder, including the step of administering to a subject or patient a therapeutically effective amount of a histone H2A polypeptide of the invention that specifically binds to a human monoclonal antibody produced by the cell line deposited as ATCC Accession No. CRL-12142 to treat the cell proliferative disorder. The cell proliferative disorder being treated may comprise a cell selected from or arising from a breast, colon, gut, lung, brain, skin or pancreas cell.

In a further aspect, the invention features a method of treating a subject having, or at risk of having, a tumor, comprising administering to the subject or patient a therapeutically effective amount of a histone H2A polypeptide of the invention that specifically binds to a human monoclonal antibody produced by the cell line deposited as ATCC accession no. CRL-12142 to treat the subject or patient.

In an embodiment of this aspect, the treatment reduces tumor volume; inhibits an increase in tumor volume; inhibits progression or metastasis of the tumor; stimulates tumor cell lysis, necrosis or apoptosis; or reduces tumor metastasis. In another embodiment of this aspect, the treatment reduces one or more adverse symptoms associated with the tumor. In further embodiment of this aspect, the treatment reduces mortality. In some embodiments of this aspect, the subject or patient is a candidate for, is undergoing, or has undergone an anti-tumor or immune system-enhancing therapy. In still further embodiments of this aspect, the method further comprises the step of administering an anti-tumor or immune system-enhancing agent or treatment.

This further step of administering an anti-tumor or immune system-enhancing agent or treatment may comprise administration of an antibody; a radioisotope; radiation; a toxic, immunotherapeutic or chemotherapeutic agent; immunotherapy; hyperthermia; or performing surgery to bring about a resection. The chemotherapeutic agent administered may comprise an alkylating agent, an anti-metabolite, a plant extract, a plant alkaloid, nitrosourea, a hormone, a nucleoside, a nucleotide analogue, cyclophosphamide, colchicine, colcemid, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC), and 5-azacytidine, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin or dibromomannitol. Other chemotherapeutic agents are listed in U.S. Pat. No. 6,608,096.

In a further aspect, the invention features a method of identifying an inhibitor or stimulator of expression of a histone H2A polypeptide of the invention that specifically binds to a monoclonal antibody produced by the cell line deposited as ATCC accession no. CRL-12142, comprising the steps of (1) contacting a cell that expresses or is capable of expressing the histone H2A polypeptide of the invention that specifically binds to a monoclonal antibody produced by the cell line deposited as ATCC accession no. CRL-12142 with a test compound; and (2) detecting expression of the aforementioned histone H2A polypeptide of the invention that specifically binds to a monoclonal antibody produced by the cell line deposited as ATCC accession no. CRL-12142, wherein a change in expression indicates that the test compound is an inhibitor or stimulator of expression of the histone H2A polypeptide of the invention that specifically binds to a monoclonal antibody produced by the cell line deposited as ATCC accession no. CRL-12142.

In this aspect, the contacting of the cell step may be in solution, in solid phase, in vivo or in vitro.

In a final aspect, the invention features a method of screening a subject having or at risk of having a cell proliferative disorder, where this cell proliferative disorder arises from a tissue selected from breast, colon, gut, lung, brain, skin or pancreas, where the method comprises the step of analyzing for the expression of an histone H2A polypeptide of the invention that migrates in denaturing gel electrophoresis at about 19 kDa and that specifically binds to a monoclonal antibody produced by the cell line deposited as ATCC accession no. CRL-12142, wherein the presence of the histone H2A polypeptide of the invention that migrates in denaturing gel electrophoresis at about 19 kDa and that specifically binds to a human monoclonal antibody produced by the cell line deposited as ATCC accession no. CRL-12142 in the tissue identifies the subject as having or being at risk of having a cell proliferative disorder.

### BRIEF DESCRIPTION OF THE DRAWINGS

FIG. 1 represents possible DNA and the protein sequences for AgRM1, i.e., a truncated H2A protein.

AgRM1 is estimated as containing 78 amino acids with a MW of 19 kDa as estimated by its mobility in denaturing gel electrophoresis. The encoding DNA sequence encoding the first depicted AgRM1 begins with GCG. This GCG encodes alanine at position 52 of this intact H2A histone molecule.

FIG. 2 represents the results of the xenograft studies using the anti-RM1 antibody to inhibit tumor growth in a xenograft model. The anti-RM1 antibody inhibits the growth of tumor cells in a mouse model.

FIG. 3 represents the results of the xenograft studies using the anti-RM1 antibody and the anti-RM2 antibody to inhibit tumor growth in a xenograft model. Values in the table reflect tumor volumes.

FIG. 4 represents the results of staining a panel of cell lines with an antibody that recognizes AgRM1. Cells were either positive (+) or negative (−) for staining.

### DETAILED DESCRIPTION OF THE INVENTION

The invention is based at least in part on the isolation and characterization of AgRM1, an antigen associated with cell proliferative disorders, such as metastatic and non-metastatic tumors. AgRM1 was identified as a subsequence of histone H2A. AgRM1 is approximately 19 kDa, as determined by denaturing gel electrophoresis. AgRM1 is expressed at least in part on the cell surface. AgRM1 is more highly expressed in proliferating cells than in non-proliferating cells. For example, AgRM1 may be associated with tumors including, for example, breast, colon, gut, lung, brain, skin and pancreas. AgRM1 is associated with the cell membranes of cell lines derived from colon cancer and lung cancer. AgRM1 is therefore useful for diagnosis, prognosis and treatment of cell proliferative disorders.

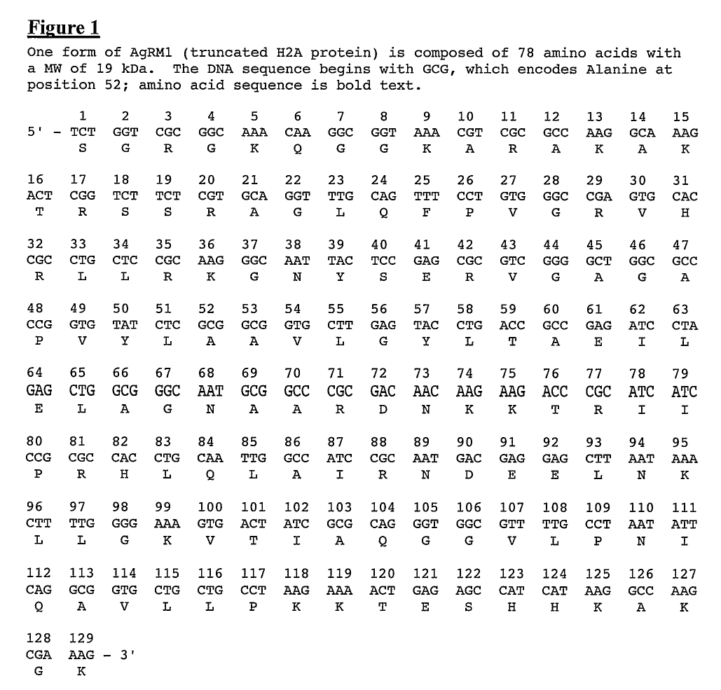

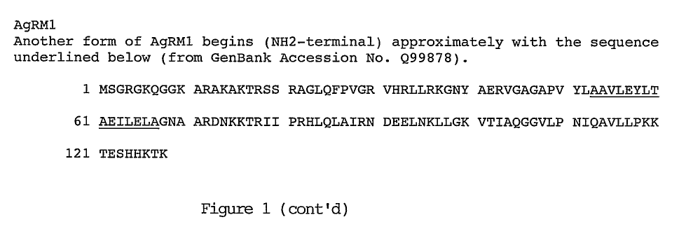

Thus, in accordance with the invention, there are provided isolated and purified subsequences of histone H2A, and nucleic acids that encode isolated and purified subsequences of histone H2A. In one embodiment, an isolated or purified histone H2A polypeptide specifically binds to a human monoclonal antibody produced by the cell line deposited as ATCC accession no. CRL-12142. In various aspects, the histone H2A polypeptide sequence has a deletion at the amino terminus relative to full length native human histone H2A (e.g., of at least about 10, 20, 30, 40, 45, 50, 51, 55, 60, 70, 80 amino acids from the amino terminus relative to full length native human histone H2A). In various additional aspects, the histone H2A polypeptide consists of a polypeptide having no more than about 50 to 70, about 70 to 90, about 90 to 100, and about 128 amino acids in length. H2A polypeptide sequences of the invention include mammalian sequences, including human (as set forth in FIG. 1), polymorphic variants thereof (e.g., polypeptides encoded by a sequence selected the group consisting of from GenBank Accession Nos. AF058445; AF058446; AF044286; AF058444; NM138609; NM138609; BC013331; AF054174; AF041483; NM138610.1; and NM004893.2), and variants of these or similar sequences. Other, non-limiting, examples of these variants are set forth in Table 1, above.

Definitions

The terms “protein,” “polypeptide” and “peptide” are used interchangeably herein to refer to two or more covalently linked amino acids, or “residues,” through an amide bond or equivalent. Polypeptides are of unlimited length. The amino acids may be linked by non-natural and non-amide chemical bonds including, for example, those formed with glutaraldehyde, N-hydoxysuccinimide esters, bifunctional maleimides, or N,N′-dicyclohexylcarbodiimide (DCC). Non-amide bonds include, for example, ketomethylene, aminomethylene, olefin, ether, thioether and the like (see, e.g., Spatola (1983) in Chemistry and Biochemistry of Amino Acids Peptides and Proteins, Vol. 7, pp 267-357, “Peptide and Backbone Modifications,” Marcel Decker, NY).

The molecular weight of proteins is frequently estimated in comparison to markers of known molecular weight in denaturing gel electrophoresis, i.e., SDS-PAGE. See e.g. the standard standard techniques of SDS-PAGE as first published by Laemmli and co-workers and as set forth in Sambrook et al., In: Molecular Cloning: A Laboratory Manual, 3^{rd} ed., Cold Spring Harbor Laboratory Press, 2001); and Ausubel et al., Current Protocols in Molecular Biology, Greene Publishing Assoc. and Wiley Interscience, N.Y., 1989.

As used herein, the term “isolated,” when used as a modifier of an invention composition (e.g., polypeptides and nucleic acids, and modified forms, subsequences, cells, and vectors thereof), means that the compositions are made by the hand of man or are separated from their naturally occurring in vivo environment. Generally, compositions so separated are substantially free of one or more materials with which they normally associate with in nature, for example, one or more protein, nucleic acid, lipid, carbohydrate, cell membrane. Thus, an isolated protein is typically substantially free of one or more materials with which it may typically associate with in nature. The term “isolated” does not exclude alternative physical forms, such as polypeptide multimers, post-translational modifications (e.g., phosphorylation, glycosylation) or derivatized forms.

An “isolated” protein can also be “purified” when free of most or all of the materials with which it typically associates with in nature. Thus, an isolated molecule that also is substantially pure does not include polypeptides or polynucleotides present among millions of other sequences, such as polypeptides of a polypeptide library or nucleic acids of a genomic or cDNA library, for example. Of course, a “purified” molecule can be combined with one or more other molecules. Thus, the term “purified” does not exclude combination compositions made by the hand of man.

Purified can be at least about 60% or more of the molecule by mass. Purity can also be about 70% or 80% or more, and can be greater, for example, about 90% or more. Purity can be determined by any appropriate method, including, for example, UV spectroscopy, chromatography (e.g., HPLC, gas phase), gel electrophoresis (e.g., silver or coomassie staining) and sequence analysis (nucleic acid and peptide).

The term “antibody” refers to a protein that binds to other molecules (antigens) via heavy and light chain variable domains, VH and VL, respectively. Antibodies include IgG, IgD, IgA, IgM and IgE, subtypes, and mixtures thereof. Antibodies may be polyclonal or monoclonal, intact immunoglobulin molecules, two full length heavy chains linked by disulfide bonds to two full length light chains, or subsequences (i.e. fragments) thereof, with our without constant region, that bind to an epitope of an antigen, and mixtures thereof. Antibodies may comprise heavy or light chain variable regions, V_{H} or V_{L} individually, or in any combination. Antibodies may be engineered to contain an antigen binding site, both V_{H} and V_{L} regions, in a single chain.

Polypeptides and nucleic acids of the invention include modified or variant forms. The term “modify” and grammatical variations thereof, when used in reference to a composition such as a polypeptide or nucleic acid, means that the modified composition deviates from a reference composition. Polypeptide modifications include amino acid substitutions, additions and deletions (subsequences and fragments), which are also referred to as “variants.” Polypeptide modifications also include one or more D-amino acids substituted for L-amino acids (and mixtures thereof), structural and functional analogues, for example, peptidomimetics having synthetic or non-natural amino acids or amino acid analogues and derivatized forms.

Polypeptide modifications further include fusion (chimeric) polypeptide sequences, which is an amino acid sequence having one or more molecules not normally present in a reference native (wild type) sequence covalently attached to the sequence, for example, one or more amino acids of non-histone H2A attached to histone H2A polypeptide that specifically binds to a human monoclonal antibody produced by the cell line deposited as ATCC accession no. CRL-12142. Such chimeric polypeptides can comprise apoptotic factors, differentiative factors, toxins, chemokines and cytokines (interleukins, interferons).

Modifications include cyclic structures such as an end-to-end amide bond between the amino and carboxy-terminus of the molecule or intra- or inter-molecular disulfide bond. Polypeptides may be modified in vitro or in vivo, e.g., post-translationally modified to include, for example, sugar residues, phosphate groups, ubiquitin, SUMA, fatty acids, acetyl groups, or lipids.

Modifications include an activity or function of a reference composition (e.g., binding to a human monoclonal antibody produced by the cell line deposited as ATCC accession no. CRL-12142). A modified protein can have an amino acid substitution, addition or deletion (e.g., 1-3, 3-5, 5-10 or more). In a particular non-limiting example, the substitution is a conservative amino acid substitution.

A “conservative substitution” means the replacement of one amino acid by a biologically, chemically or structurally similar residue. Biologically similar means that the substitution is compatible with biological activity, e.g., binds to a human monoclonal antibody produced by the cell line deposited as ATCC accession no. CRL-12142. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

Modified polypeptides may include amino acid sequence with about 50%, 60%, 70%, 75%, 80%, 85%, 90%, 95%, 96%, 97%, 98%, or more identity to a sequence of H2A as set forth in FIG. 1. The identity can be over a defined area of the protein, such as 50, 60, 75, 76, 77, 78, 79, 80, 85 90 or more amino acids.

The term “identity” and grammatical variations thereof, mean that two or more referenced entities are the same. Thus, where two protein sequences are identical, they have the same amino acid sequence. “Areas of identity” means that a portion of two or more referenced entities are the same. Thus, where two protein sequences are identical over one or more sequence regions they share identity in these regions. The term “substantial identity” means that the identity is structurally or fimctionally significant. That is, the identity is such that the molecules are structurally identical or have at least one of the same fimctions (e.g., biological function) even though the molecules are different.

Due to variation in the amount of sequence conservation between structurally and fimctionally related proteins, the amount of sequence identity for substantial identity will depend upon the type of protein, the region and any function. There can be as little as 30% sequence identity for proteins to have substantial identity, but typically there is more, e.g., about 50%, 60%, 75%, 85%, 90%, 95%, 96%, 97%, 98%, identity to a reference sequence. For nucleic acid sequences, 50% sequence identity or more typically constitutes substantial homology, but again can vary depending on the comparison region and its function, if any.

The extent of identity between two sequences can be ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al. (1990) J. Mol. Biol. 215:403-10, publicly available through NCBI) has exemplary search parameters as follows: Mismatch -2; gap open 5; gap extension 2. For polypeptide sequence comparisons, a BLASTP algorithm is typically used in combination with a scoring matrix, such as PAM100, PAM 250, and BLOSUM 62.

As used herein, the term “subsequence” or “fragment” means a portion of the full length molecule. For example, a subsequence of H2A is at least one amino acid less in length than full length H2A (e.g. one or more internal or terminal amino acid deletions from either amino or carboxy-termini). Subsequences therefore can be any length up to, but not including, the full length molecule.

Subsequences include portions which retain at least part of the function or activity of a full length sequence. Subsequences may also acquire a function or activity absent from a full length sequence. For example, a protein subsequence may display an epitope that is absent from or hidden in a full length sequence. In particular, an invention isolated or purified H2A polypeptide binds to a human monoclonal antibody produced by the cell line deposited as ATCC accession no. CRL-12142, whereas full-length wild type H2A does not exhibit detectable specific binding to monoclonal antibody produced by the cell line deposited as ATCC accession no. CRL-12142.

Modified polypeptides and nucleic acids can include one or more non-native (wild-type) functions, or be “multifunctional,” which means that the composition referred to has one or more different or additional activities or functions. Particular non-limiting examples include, for example, enzyme activity, ligand or receptor binding (substrates, agonists and antagonists), detection, purification, and toxicity.

A particular example of an additional function is a “detectable label,” which refers to a molecule that enables detection of the conjugated molecule. Examples of detectable labels include chelators, photoactive agents, radioisotopes and radionuclides (alpha, beta and gamma emitters), fluorescent agents and paramagnetic ions. Radioactive labels include, for example, ³H, ¹⁴C, ³²P, ³³P, 35S, ¹²⁵I, ¹³¹I, non-radioactive moieties such as gold particles, colored glass or plastic polystyrene, polypropylene, or latex beads, and amino acid sequences such as tags, as set forth herein. Detectable labels include enzyme activity (e.g., green fluorescent protein, acetyltransferase, galactosidase, glucose oxidase, peroxidase, horseradish peroxidase (HRP), urease and alkaline phosphatase). Detectable fluorescent compounds include, for example, fluorescein isothiocyanate, rhodamine, phycoerytherin, phycocyanin, allophycocyanin, o-phthaldehyde, fluorescamine, and commercially available fluorophores such as Alexa Fluor 350, Alexa Fluor 488, Alexa Fluor 532, Alexa Fluor 546, Alexa Fluor 568, Alexa Fluor 594, Alexa Fluor 647, and BODIPY dyes such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, D ansyl, lissamine rhodamine B, Marina Blue, Oregon Green 488, Oregon Green 514, Pacific Blue, rhodamine 6G, rhodamine green, rhodamine red, tetramethylrhodamine and Texas Red, from Molecular Probes, Inc., Eugene, Oreg.), colloidal metals, quantum dots, chemiluminescent compounds (e.g., luminol, isoluminol, an aromatic acridinium ester, an imidazole, an acridinium salt and oxalate esters), bioluminescent compounds (e.g., luciferin, luciferase and aequorin), paramagnetic labels (e.g., chromium (III), manganese (II), manganese (III), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III)) which can be detected by MRI, and adhesion proteins (e.g., biotin, streptavidin, avidin, and other lectins).

Another particular example of an additional function is a “tag,” which refers to a molecule conjugated to another that allows detection, isolation or purification. Specific examples of tags include immunoglobulins, T7, polyhistidine tags, glutathione-S-transferase, a chitin-binding tag, calmodulin-binding tag, myc tag, biotin tag, avidin tag, and a Xpress epitope (detectable by anti-Xpress antibody; Invitrogen, Carlsbad, Calif., USA).

Additional candidate modified forms include added cytotoxicity functions (e.g., bacterial cholera toxin, pertussis toxin, anthrax toxin lethal factor, Pseudomonas exotoxin A, diphtheria toxin, plant toxin ricin, radionuclides such as ⁴⁷SC ⁶⁷Cu, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At, ²¹²Bi, ²¹³Bi, ²¹²Pb, ²²³Ra, ²²⁵AC, ²²⁷AC, ²²⁸Th, and cytotoxic drugs). Modified polypeptides and nucleic acids therefore also include addition of functional entities, covalently or non-covalently attached to the polypeptides and nucleic acids of the invention.

Polypeptides, including modified forms such as substitutions, additions, and deletions (e.g., subsequences and fragments), can be produced using recombinant technology of polypeptide encoding nucleic acids via cell expression or in vitro translation. Polypeptides including antibodies can also be produced by a chemical synthesizer (see, e.g., Applied Biosystems, Foster City, Calif.). Antibodies can be expressed from recombinantly produced antibody-encoding nucleic acid (see, e.g., Harlow and Lane, Using Antibodies: A Laboratory Manual, Cold Spring Harbor Laboratory, 1999; Fitzgerald et al., J.A.C.S. 117:11075 (1995); Gram et al., Proc. Natl. Acad. Sci. USA 89:3576 (1992)). Enzymes such proteases (e.g., pepsin and papain) can be used to generate subsequences and fragments.

The invention provides nucleic acids encoding and vectors containing invention polypeptides, including modified forms of H2A. Also provided are transformed cells that contain H2A polypeptide of the invention. Transformed cells including nucleic acid encoding H2A polypeptide of the invention are further provided. Transformed cells include prokaryotic and eukaryotic cells, such as bacteria, fungi, insect and mammalian cells in culture, ex vivo or in vivo.

As used herein, “nucleic acid,” refers to at least two or more ribo- or deoxy-ribonucleic acid base pairs (nucleotides) linked through a phosphoester bond or equivalent. Nucleic acids include polynucleotides and polynucleosides. Nucleic acids include circular and linear, single, double and triplex molecules. A nucleic acid molecule may belong exclusively to or be in a mixture, but not limited to: RNA, DNA, cDNA, genomic nucleic acid, non-genomic nucleic acid, naturally occurring and non naturally occurring nucleic acid and synthetic nucleic acid.

Nucleic acids can be of any length, and typically range from about 20 nucleotides to about 10 Kb, about 10 nucleotides to about 5,000, about 1 Kb to about 5 Kb or less, about 1000 to about 500 nucleotides or less in length. Nucleic acids can also be shorter, for example, about 100 to about 500 nucleotides, or from about 12 to about 25, about 25 to about 50, about 50 to about 100, about 100 to about 250, or about 250 to about 500 nucleotides in length.

Nucleic acids further include modifications such as nucleotide and nucleoside substitutions, additions and deletions, as well as derivatized forms and fusion sequences (e.g., encoding chimeric H2A polypeptide).

For example, due to the degeneracy of the genetic code, nucleic acids include sequences and subsequences that are degenerate with respect to nucleic acids that encode H2A as set forth in FIG. 1. Other examples are nucleic acids complementary to a sequence that encodes H2A as set forth in FIG. 1. Nucleic acid deletions (subsequences) have from about 10 to about 25, about 25 to about 50, about 50 to about 100, about 100 to about 200, about 200 to about 300, about 300 to about 500, about 500 to about 1000 nucleotides, or more. Such nucleic acids are useful for expressing H2A polypeptide fragments, for genetic manipulation (as primers and templates for PCR amplification), and as probes to detect the presence or an amount of a sequence encoding an H2A histone polypeptide of the invention in vitro, in a cell, culture medium, biological sample (e.g., tissue, organ, blood or serum), or in a subject or patient in vivo.

Nucleic acids that hybridize at high stringency to nucleic acids that encode an H2A polypeptide, a subsequence thereof and nucleic acid sequences complementary to the encoding nucleic acids, are provided. Hybridizing nucleic acids are useful for detecting the presence or an amount of a sequence encoding an H2A histone polypeptide of the invention in vitro, or in a cell, culture medium, biological sample (e.g., tissue, organ, blood or serum), or in a subject or patient in vivo.

The term “hybridize” refers to the binding between nucleic acid sequences. Hybridizing sequences will generally have more than about 50% homology to a nucleic acid that encodes an amino acid sequence of RM4.

The hybridization region between hybridizing sequences can extend over at least about 10 to about 15 nucleotides, about 15 to about 20 nucleotides, about 20 to about 30 nucleotides, about 30 to about 50 nucleotides, about 50 to about 100 nucleotides, or about 100 to about 200 nucleotides or more.

As is understood by one skilled in the art, the T_{M} (melting temperature) is the temperature at which binding between two nucleic acid sequences is no longer stable. For two sequences to bind, the temperature of a hybridization reaction must be less than the calculated T_{M} for the sequences under the hybridization conditions. The T_{M} is influenced by the amount of sequence complementarity, length, composition (% GC), type of nucleic acid (RNA vs. DNA), and the amount of salt, detergent and other components in the reaction (e.g., formamide). All of these factors are considered in establishing appropriate hybridization conditions (see, e.g., the hybridization techniques and formula for calculating T_{M} described in Sambrook et al., In: Molecular Cloning: A Laboratory Manual, 3^{rd} ed., Cold Spring Harbor Laboratory Press, 2001). See also Ausubel et al., Current Protocols in Molecular Biology, Greene Publishing Assoc. and Wiley Interscience, N.Y., 1989.

Typically, wash conditions are adjusted to attain the desired degree of hybridization stringency. Thus, hybridization stringency can be determined empirically, for example, by washing under particular conditions, e.g., at low stringency conditions or high stringency conditions. Optimal conditions for selective hybridization will vary depending on the particular hybridization reaction involved. An example of high stringency hybridization conditions are as follows: 2×SSC/0.1% SDS at about 37° C. or 42° C. (hybridization conditions); 0.5×SSC/0.1% SDS at about room temperature (low stringency wash); 0.5×SSC/0.1% SDS at about 42° C. (moderate stringency wash); and 0.1×SSC/0.1% SDS at about 65° C. (high stringency wash).

Nucleic acids can be produced using various standard cloning and chemical synthesis techniques. Such techniques include nucleic acid amplification, e.g., polymerase chain reaction (PCR), with genomic DNA or cDNA targets using primers (e.g., a degenerate primer mixture) capable of annealing to the H2A encoding sequence; and chemical synthesis of nucleic acid sequences. The sequences produced can then be translated in vitro, or cloned into a plasmid and propagated and then expressed in a cell (e.g., microorganism, such as yeast or bacteria, a eukaryote such as an animal or mammalian cell or in a plant).

The invention further provides expression cassettes including a nucleic acid encoding an H2A histone polypeptide of the invention operably linked to an expression control element. The term “operably linked” refers to a physical or functional relationship between the elements referred to that permit them to operate in their intended fashion. Thus, an expression control element “operably linked” to a nucleic acid means that the control element modulates nucleic acid transcription and, as appropriate, translation of the transcript.

Physical linkage is not required for the elements to be operably linked. For example, a minimal element can be linked to a nucleic acid encoding an H2A histone polypeptide of the invention. A second element that controls expression of an operably linked nucleic acid encoding a protein that functions “in trans” to bind to the minimal element can influence expression of the H2A polypeptide. Because the second element regulates expression of H2A polypeptide, the second element is operably linked to the nucleic acid encoding the H2A polypeptide even though it is not physically linked.

The term “expression control element” refers to nucleic acid that influences expression of an operably linked nucleic acid. Promoters and enhancers are particular non-limiting examples of expression control elements. A “promoter sequence” is a regulatory region capable of initiating transcription of a downstream (3′ direction) coding sequence. The promoter sequence includes nucleotides for facilitating transcription initiation. Enhancers also regulate gene expression, but can function a distance from the transcription start site of the gene to which it is operably linked (e.g., at either 5′ or 3′ ends of the gene, as well as within the gene). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, and stop codons.

Expression control elements include “constitutive” elements such that transcription of the operably linked nucleic acid occurs without a signal or stimuli. Expression control elements that confer expression in response to a signal or stimuli, which either increases or decreases expression of the operably linked nucleic acid, are “regulatable.” A regulatable element that increases expression of the operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, and are referred to as “tissue-specific” expression control elements. Tissue-specific expression control elements are typically active in specific cell or tissue types because they are recognized by transcriptional activator proteins, or other regulators of transcription, that are unique to the specific cell or tissue type.

Expression control elements include full-length nucleic acid sequences, such as native promoter and enhancer elements, as well as subsequences or nucleotide variants thereof (e.g., substituted/mutated or other forms that differ from native sequences) which retain all or part of full-length or non-variant control element function (confer regulation, e.g., retain some amount of inducibility in response to a signal or stimuli).

For bacterial expression, constitutive promoters include T7, as well as inducible promoters such as pL of bacteriophage λ, plac, ptrp, ptac (ptrp-lac hybrid promoter). In insect cell systems, constitutive or inducible promoters (e.g., ecdysone) may be used. Other examples of insect cell promoters include the p10 promter and the polyhedrin promoter. In yeast, constitutive promoters include, for example, ADH or LEU2 and inducible promoters such as GAL (see, e.g., Ausubel et al., In: Current Protocols in Molecular Biology, Vol. 2, Ch. 13, ed., Greene Publish. Assoc. & Wiley Interscience, 1988; Grant et al., (1987) In: Methods in Enzymology, 153:516-544, eds. Wu & Grossman, 1987, Acad. Press, N.Y.; Glover, DNA Cloning, Vol. 11, Ch. 3, IRL Press, Wash., D.C., 1986; Bitter (1987) In: Methods in Enzymology, 152:673-684, eds. Berger & Kimmel, Acad. Press, N.Y.; Strathern et al., The Molecular Biology of the Yeast Saccharomyces (1982) eds. Cold Spring Harbor Press, Vols. I and II; and Sambrook et al., Molecular Cloning: A Laboratory Manual, 3^{rd} ed., Cold Spring Harbor Laboratory Press, 2001).

For mammalian expression, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; the inducible mouse mammary tumor virus LTR) are used.

The invention also provides stably and transiently transformed cells and progeny thereof, where progeny includes all descendent cells, into which a nucleic acid molecule encoding an H2A histone polypeptide of the invention has been introduced by means of recombinant DNA techniques in vitro, ex vivo or in vivo. The transformed cells can be propagated and the introduced nucleic acid transcribed, or encoded protein expressed. Transformed cells include but are not limited to prokaryotic and eukaryotic cells such as bacteria, fungi, plant, insect, and animal (e.g., mammalian, including human) cells. The cells may be present in culture, in tissue or an organ ex vivo, or in a subject or patient. A progeny cell may not be identical to the parental cell, since there may be mutations that occur during parental cell replication.

The term “transformed” means a change in a cell following incorporation of nucleic acid (e.g., a transgene) or protein exogenous to the cell. Thus, “transformed cells” include cells into which, or a progeny of which, a nucleic acid or polypeptide has been introduced by means of recombinant DNA techniques. Cell transformation to produce such cells may be carried out as described herein or using techniques known in the art. Accordingly, methods of producing cells including the nucleic acids and polypeptides of the invention are also provided.

Typically, cell transformation with a nucleic acid employs a “vector,” which refers to a plasmid, virus, such as a viral vector, or other vehicle known in the art that can be manipulated by insertion or incorporation of a nucleic acid sequence. For genetic manipulation “cloning vectors” can be employed, and to transcribe or translate the inserted polynucleotide “expression vectors” can be employed. Such vectors are useful for introducing nucleic acids, including a nucleic acid that encodes an H2A polypeptide operably linked with an expression control element, and expressing the H2A polypeptide in vitro (e.g., in solution or in solid phase), in cells or in a subject or patient in vivo.

A vector generally contains an origin of replication for propagation in a cell. Control elements, including expression control elements as set forth herein, present within a vector, can be included to facilitate transcription and translation.

Vectors can include a selection marker, which is a gene that allows for the selection of cells containing the gene. “Positive selection” refers to a process whereby only cells that contain the selection marker will be selected. Drug resistance is one example of a positive selection marker; cells containing the marker will survive in culture medium containing the selection drug, and cells lacking the marker will die. Selection markers include drug resistance genes such as neo, which confers resistance to G418; hygr, which confers resistance to hygromycin; and puro which confers resistance to puromycin. Other positive selection marker genes include genes that allow identification or screening of cells containing the marker. These genes include genes for fluorescent proteins (GFP and GFP-like chromophores, luciferase), the lacZ gene, the alkaline phosphatase gene, and surface markers such as CD8, among others. “Negative selection” refers to a process whereby cells containing a negative selection marker are not selected, for example, due to exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine. Vectors can also include an amplification marker such as the gene providing resistance to methotrexate (see e.g. U.S. Pat. No. 5,179,017), or the CAD gene (see, e.g., Wahl et al., Somat. Cell Mol. Genet. 12:339 (1986).

Viral vectors included are those based on retroviral, adeno-associated virus (AAV), adenovirus, reovirus, lentivirus, rotavirus genomes, simian virus 40 (SV40) or bovine papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981)). Additional viral vectors useful for expression include parvovirus, rotavirus, Norwalk virus, coronaviruses, paramyxo and rhabdoviruses, togavirus (e.g., sindbis virus and semliki forest virus) and vesicular stomatitis virus (VSV).

Mammalian expression vectors include those designed for in vivo and ex vivo expression, such as AAV (U.S. Pat. No. 5,604,090). AAV vectors have previously been shown to provide expression of Factor IX in humans and in mice at levels sufficient for therapeutic benefit (Kay et al., Nat. Genet. 24:257 (2000); Nakai et al., Blood 91:4600 (1998)). Adenoviral vectors (U.S. Pat. Nos. 5,700,470, 5,731,172 and 5,928,944), herpes simplex virus vectors (U.S. Pat. No. 5,501,979) retroviral (e.g., lentivirus vectors are useful for infecting dividing as well as non-dividing cells and foamy virues) vectors (U.S. Pat. Nos. 5,624,820, 5,693,508, 5,665,577, 6,013,516 and 5,674,703 and WIPO publications WO92/05266 and WO92/14829) and papilloma virus vectors (e.g., human and bovine papilloma virus) have all been employed in gene therapy (U.S. Pat. No. 5,719,054). Vectors also include cytomegalovirus (CMV) based vectors (U.S. Pat. No. 5,561,063). Vectors that efficiently deliver genes to cells of the intestinal tract have been developed (see, e.g., U.S. Pat. Nos. 5,821,235, 5,786,340 and 6,110,456).

Introduction of polypeptides and nucleic acids into target cells can also be carried out by methods known in the art such as osmotic shock (e.g., calcium phosphate), electroporation, microinjection, cell fusion, etc. Introduction of polypeptides and nucleic acids in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. Polypeptides and nucleic acids can be entrapped in microcapsules prepared by coacervation techniques or by interfacial polymerization, for example, by the use of hydroxymethylcellulose or gelatin-microcapsules, or poly(methylmethacrolate)microcapsules, respectively, or in a colloid drug delivery system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells, including polypeptides and nucleic acids, are known to one skilled in the art and include, for example, phosphatidylcholine, phosphatidylserine, lipofectin and DOTAP, (see, e.g., U.S. Pat. Nos. 4,844,904, 5,000,959, 4,863,740, 4,975,282, GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Accordingly, viral and non-viral vector means of delivery into cells or tissue, in vitro, in vivo and ex vivo are included.

H2A histone polypeptides of the invention and nucleic acids can be combined with any other compound or agent that provides an enhanced or synergistic therapeutic benefit. The invention therefore also provides combination compositions including an H2A histone polypeptide of the invention or an nucleic acid encoding a H2A histone of the invention and one or more additional compounds or agents and methods of using the combinations. For example, an H2A histone polypeptide of the invention may be combined with a compound or agent that has anti-cell proliferative (e.g., anti-tumor) activity or immune system-enhancing activity.

As used here, the term “anti-cell proliferative activity,” when used in reference to a compound, agent, therapy or treatment, means that the compound, agent, therapy or treatment, reduces or inhibits cell proliferation or growth, stimulates or promotes cell apoptosis, lysis, necrosis or differentiation. The term “immune system-enhancing,” when used in reference to a compound, agent, therapy or treatment, means that the compound, agent, therapy or treatment, provides an increase, stimulation, induction or promotion of an immune response, humoral or cell-mediated. Agents or compositions which bring about an enhanced immune response are variously referred to as “vaccines” or “immunogens.” Such anti-cell proliferative and immune system-enhancing therapies and treatments can reduce or inhibit cell proliferation or enhance immune response generally, or reduce or inhibit cell proliferation of, or enhance immune response towards, a specific target, such as a cell proliferative disorder (e.g., a tumor).

Specific non-limiting examples of anti-cell proliferative and immune system-enhancing agents include monoclonal, polyclonal antibody and mixtures thereof. Antibodies include antibodies that bind to tumor-associated antigens (TAA). A “tumor associated antigen” or “TAA” refers to an antigen expressed by a tumor cell. TAAs may be expressed in amounts greater in tumor cells than a normal non-tumor cell counterpart, or may be expressed at similar levels, or at levels less than a normal cell counterpart.

Particular non-limiting examples of TAAs that can be targeted and TAA binding antibodies include, for example, human IBD 12 monoclonal antibody which binds to epithelial cell surface H antigen (U.S. Pat. No. 4,814,275); M195 antibody which binds to leukemia cell CD33 antigen (U.S. Pat. No. 6,599,505); monoclonal antibody DS6 which binds to ovarian carcinoma CA6 tumor-associated antigen (U.S. Pat. No. 6,596,503); and BR96 antibody which binds to Le^{x} carbohydrate epitope expressed by colon, breast, ovary, and lung carcinomas. Additional antibodies having anti-tumor activity that can be employed include, for example, Rituxan®, Herceptin (anti-Her-2 neu antibody), Bevacizumab (Avastin), Zevalin, Bexxar, Oncolym, 17-1A (Edrecolomab), 3F8 (anti-neuroblastoma antibody), MDX-CTLA4, Campath®, Mylotarg and IMC-C225 (Cetuximab).

Other non-limiting examples of TAAs that can be targeted include MUC-1, HER-2/neu, MAGE, p53, T/Tn and CEA (breast cancer); MUC-2 and MUC-4, CEA, p53 and the MAGE (colon cancer); MAGE, MART-1 and gp100 (melanoma); GM2, Tn, sTn, Thompson-Friedenreich antigen (TF), MUC1, MUC2, beta chain of chorionic gonadotropin (hCG beta), HER2/neu, PSMA and PSA (prostate cancer); chorionic gonadotropin (testicular cancer); and alpha fetoprotein (hepato-cellular carcinoma), as well as EGF.

Additional examples of immune system-enhancing agents include immune cells such as lymphocytes, plasma cells, macrophages, NK cells and B-cells expressing antibody against the tumor. Cytokines that enhance or stimulate immunogenicity against tumor such as IL-2, IL-1α, IL-1β, IL-3, IL-6, IL-7, granulocyte-macrophage-colony stimulating factor (GMCSF), IFN-γ, IL-12, TNF-α, and TNFβ are also non-limiting examples of immune system-enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAPIIIβ-TG/NAP-2, Mig, PBSF/SDF-1, and lymphotactin are additional non-limiting examples of immune system-enhancing agents.

An “anti-tumor,” “anti-cancer” or “anti-neoplastic” treatment, therapy, activity or effect refers to any compound, agent, therapy or treatment regimen or protocol that inhibits, decreases, slows, reduces or prevents hyperplastic, tumor, cancer or neoplastic growth, metastasis, proliferation or survival. Anti-tumor compounds, agents, therapies or treatments can operate by disrupting, inhibiting or delaying cell cycle progression or cell proliferation; stimulating or enhancing apoptosis, lysis or cell death or necrosis; stimulating or enhancing cell differentiation; inhibiting nucleic acid or protein synthesis or metabolism; and inhibiting cell division, or decreasing, reducing or inhibiting cell survival, or production or utilization of a necessary cell survival factor, growth factor or signaling pathway (extracellular or intracellular). Examples of anti-tumor therapy include chemotherapy, immunotherapy, radiotherapy (ionizing or chemical), local thermal (hyperthermia) therapy and surgical resection.

Specific non-limiting examples of agent classes having anti-cell proliferative and anti-tumor activities include alkylating agents, anti-metabolites, plant extracts, plant alkaloids, nitrosoureas, hormones, nucleoside and nucleotide analogues. Specific examples of drugs having anti-cell proliferative and anti-tumor activities include cyclophosphamide, colchicine, colcemid, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol. Other examples of chemotherapeutic agents are listed in U.S. Pat. No. 6,608,096.

The term “treating” refers to administering a composition to an organism afflicted with an abnormal condition, such as a cell proliferative disorder, where the administration of the composition has a therapeutic effect and at least partially alleviates or abrogates the abnormal condition. Note that the treatment need not provide a complete cure and the treatment will be considered effective if at least one symptom is improved or eradicated. The treatment may reduce mortality. Furthermore, the treatment need not provide a permanent improvement of the medical condition or other abnormal condition, such as a cell proliferative disorder, although this is preferable. The treatment may work by killing the cancerous cells, facilitating the killing of the cancerours cells, inhibiting the growth of the cancerous cells, and/or inhibiting the process of the metastasis of the cancer. The treatment may also work by reducing the tumor volume, inhibiting an increase in tumor volume, inhibiting the progression of the tumor, stimulating tumor cell lysis, stimulating tumor cell lysis necrosis, or stimulating tumor cell lysis apoptosis.

The term “patient” refers to a living subject who has presented at a clinical setting with a particular symptom or symptoms suggesting the need for treatment with a therapeutic agent. The treatment may either be generally accepted in the medical community or it may be experimental. In some embodiments, the patient is a mammal, including animals such as dogs, cats, pigs, cows, sheep, goats, horses, rats, and mice. In preferred embodiments, the patient is a human. A patient's diagnosis can alter during the course of disease progression, either spontaneously or during the course of a therapeutic regimen or treatment.

The term “contacting” as used herein refers to adding together a solution or composition comprising the test compound with a liquid medium bathing the H2A histone polypeptide of invention or a cell comprising the polypeptide of the invention. The solution comprising the compound may also comprise another component, such as dimethyl sulfoxide (DMSO). DMSO facilitates the uptake of the test compound or compounds into the cells of the methods. The solution comprising the test compound may be added to the medium bathing the cells by utilizing a delivery apparatus, such as a pipette-based device or syringe-based device.

The invention further provides kits including one or more H2A polypeptides and nucleic acids of the invention, including pharmaceutical formulations, packaged into suitable packaging material. In one embodiment, a kit includes an H2A polypeptide.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., diagnosing a cell-proliferative disorder, detecting a cell-proliferative disorder, treating a cell-proliferative disorder, etc. Kits of the invention therefore can additionally include instructions for using the kit components in a method.

Thus, in additional embodiments, a kit includes a label or packaging insert including instructions for expressing an H2A histone polypeptide of the invention or a nucleic acid encoding an H2A histone polypeptide of the invention in cells in vitro, in vivo, or ex vivo. In yet additional embodiments, a kit includes a label or packaging insert including instructions for treating a subject or patient (e.g., a subject or patient having or at risk of having a cell proliferative disorder such as a tumor) with an H2A histone polypeptide of the invention or a nucleic acid encoding an H2A histone polypeptide of the invention in vivo, or ex vivo. In further embodiments, a kit includes a label or packaging insert including instructions for detecting the presence of, or the expression level of, AgRM1 in vitro or in vivo (e.g., to indicate or diagnose, or to provide a prognosis for, a subject or patient having or at risk of having a cell proliferative disorder).

Instructions can therefore include instructions for practicing any of the methods of the invention described herein. For example, invention pharmaceutical compositions can be included in a container, pack, or dispenser together with instructions for administration to a subject or patient. Instructions may additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include control components for assaying for activity, e.g., a control sample or a standard. Each component of the kit can be enclosed within an individual container or in a mixture and all of the various containers can be within single or multiple packages.

H2A polypeptides and nucleic acids of the invention, including modified forms, can be incorporated into pharmaceutical compositions. Such pharmaceutical compositions are useful for administration to a subject or patient in vivo or ex vivo, and for providing therapy for a physiological disorder or condition treatable with an H2A histone polypeptide of the invention or nucleic acid, e.g., a cell-proliferative disorder (tumor) of the breast, colon, gut, brain, lung, skin or pancreas.

Pharmaceutical compositions include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

Pharmaceutical compositions can be formulated to be compatible with a particular local or systemic route of administration. Thus, pharmaceutical compositions include carriers, diluents, or excipients suitable for administration by particular routes. Specific non-limiting examples of routes of administration for compositions of the invention are parenteral, e.g., intravenous, intradermal, intramuscular, subcutaneous, oral, transdermal (topical), transmucosal, and rectal administration.

Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate and gelatin can prolong absorption of injectable compositions.

Sterile injectable solutions can be prepared by incorporating the active compound(s) in the required amount in an appropriate solvent with one or a combination of above ingredients followed by filtered sterilization. Generally, dispersions are prepared by incorporating the active compound into a sterile vehicle containing a basic dispersion medium and other ingredients as above. In the case of sterile powders for the preparation of sterile injectable solutions, methods of preparation include, for example, vacuum drying and freeze-drying which yields a powder of the active ingredient plus any additional desired ingredient from a previously sterile-filtered solution thereof.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

H2A histone polypeptides of the invention and nucleic acids encoding them, including modified forms, can be prepared with carriers that protect against rapid elimination from the body, such as a controlled release formulation or a time delay material such as glyceryl monostearate or glyceryl stearate. The compositions can also be delivered using implants and microencapsulated delivery systems to achieve local or systemic sustained delivery or controlled release.

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known to those skilled in the art, for example, as described in U.S. Pat. No. 4,522,811.

Additional pharmaceutical formulations appropriate for administration are known in the art (see, e.g., Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Pharmaceutical Principles of Solid Dosage Forms, Technonic Publishing Co., Inc., Lancaster, Pa., (1993)).

The pharmaceutical formulations can be packaged in dosage unit form for ease of administration and uniformity of dosage. “Dosage unit form” as used herein refers to physically discrete units suited as unitary dosages treatment; each unit containing a predetermined quantity of active compound(s) in association with a pharmaceutical carrier or excipient, calculated to produce a desired therapeutic effect.

The invention also provides methods of detecting a histone H2A polypeptide that specifically binds to a human monoclonal antibody produced by the cell line deposited as ATCC accession no. CRL-12142, as well as methods of identifying cells that express a histone H2A polypeptide that specifically binds to a human monoclonal antibody produced by the cell line deposited as ATCC accession no. CRL-12142. In one embodiment, a method includes: screening a sample or a cell for the presence of a histone H2A polypeptide that specifically binds to a human monoclonal antibody produced by the cell line deposited as ATCC accession no. CRL-12142. In various aspects, the screening is performed by detecting the presence of the histone H2A polypeptide, or nucleic acid that encodes the histone H2A polypeptide. The cell may be in vitro or in a subject or patient (e.g., a mammal such as a human).

The invention further provides methods of screening for the presence of a cell proliferative disorder. In one embodiment, a method includes analyzing a biological sample for the presence of a histone H2A polypeptide that specifically binds to a human monoclonal antibody produced by the cell line deposited as ATCC accession no. CRL-12142. The presence of a histone H2A polypeptide that specifically binds to a human monoclonal antibody produced by the cell line deposited as ATCC accession no. CRL-12142 indicates the presence of a histone H2A polypeptide that specifically binds to a human monoclonal antibody produced by the cell line deposited as ATCC accession no. CRL-12142 in the subject or patient or patient. In various aspects, the screening is performed by detecting the presence of the histone H2A polypeptide, or nucleic acid that encodes the histone H2A polypeptide. The cell may be in vitro or in a subject or patient (e.g., a mammal such as a human), located in any tissue or organ. Because AgRM1 can be measured, the invention further provides methods for detecting levels or amounts of AgRM1.

Cell proliferative disorders screened or identified include benign hyperplasias and tumors, as set forth herein and known in the art. Tumors can be non-metastatic or metastatic; be in any stage (e.g., a stage I, II, III, IV or V tumor); be a solid (e.g., sarcoma, carcinoma, melanoma, myeloma, blastoma, glioma, lymphoma or leukemia) or liquid (e.g., reticuloendothelial or hematopoetic) tumor. Specific examples of cell proliferative disorders may include cells are selected from or arise from breast, colon, gut, lung, brain, skin or pancreas.

The invention moreover provides methods of inducing or increasing an immune response to a histone H2A polypeptide that specifically binds to a human monoclonal antibody produced by the cell line deposited as ATCC accession no. CRL-12142. In one embodiment, a method includes administering to a subject or patient a therapeutically effective amount of a histone H2A polypeptide that specifically binds to a human monoclonal antibody produced by the cell line deposited as ATCC accession no. CRL-12142 to elicit an immune response to the histone H2A polypeptide in the subject or patient. An immune response can include a cell-mediated or humoral immune response.

The cell may be present in a subject or patient, for example, a mammal (e.g., human subject) having or at risk of having a cell-proliferative disorder. Thus, the invention also provides methods of treating a cell-proliferative cell disorder in a subject or patient, wherein at least a portion of the cells express AgRM1. In one embodiment, a method includes administering to a subject or patient a therapeutically effective amount of a histone H2A polypeptide that specifically binds to a human monoclonal antibody produced by the cell line deposited as ATCC accession no. CRL-12142 to treat the cell proliferative disorder. Exemplary cell proliferative disorders comprise cells selected or arising from breast, colon, gut, lung, brain, skin or pancreas.

As used herein, the term “proliferate,” and grammatical variations thereof, when used in reference to a cell, tissue or organ, refers to undesirable, excessive or abnormal cell, tissue or organ proliferation, differentiation or survival. Cell proliferative and differentiative disorders include diseases and physiological conditions, both benign and neoplastic, characterized by undesirable, excessive or abnormal cell numbers, cell growth or cell survival in a subject. Specific examples of such disorders include metastatic and non-metastatic tumors and cancers.

Further provided are methods of treating a subject having or at risk of having a tumor. In one embodiment, a method includes administering to a subject or patient a therapeutically effective amount of a histone H2A polypeptide that specifically binds to a human monoclonal antibody produced by the cell line deposited as ATCC accession no. CRL-12142 to treat the subject. In additional embodiment, a method includes administering an anti cell-proliferative or immune system-enhancing treatment or therapy (e.g., an antibody, radioisotope, radiation, a toxic, immunotherapeutic or chemotherapeutic agent, immunotherapy, surgical resection, hyperthermia, vaccine or immunogen), as set forth herein and known to one skilled in the art.

The terms “tumor,” “cancer,” “malignancy,” and “neoplasia” are used interchangeably herein and refer to a cell or population of cells of any cell or tissue origin, whose growth, proliferation or survival is greater than growth, proliferation or survival of a normal counterpart cell, e.g. a cell proliferative or differentiative disorder. Such disorders include, for example, sarcoma, carcinoma, melanoma, myeloma, blastoma, neural (e.g., glioma), and reticuloendothelial or haematopoietic neoplastic disorders (e.g., myeloma, lymphoma or leukemia). Tumors can arise from a multitude of primary tumor types, including but not limited to breast, lung, thyroid, head and neck, brain, lymphoid, gut or gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, and may metastasize to secondary sites. Tumors can be non-metastatic or metastatic, and be in any stage, e.g., a stage I, II, III, IV or V tumor, or in remission.

A “solid tumor” refers to neoplasia or metastasis that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Melanoma refers to malignant tumors of melanocytes and other cells derived from pigment cell origin that may arise in the skin, the eye (including retina), or other regions of the body, including the cells derived from the neural crest that also gives rise to the melanocyte lineage. Additional carcinomas can form from the uterine/cervix, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma

A “liquid tumor” refers to neoplasia of the reticuloendothelial or haematopoetic system, such as a lymphoma, myeloma, or leukemia, or a neoplasia that is diffuse in nature. Particular examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia.

Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Methods of the invention include providing a detectable or measurable improvement in the subjects condition: i.e., a therapeutic benefit. A therapeutic benefit is any objective or subjective for a transient period of time, temporary period of time, or any longer term improvement in the condition; or a reduction in the severity or adverse symptom of the condition. Thus, a satisfactory clinical endpoint is achieved when there is an incremental or a partial reduction in the severity, duration or frequency of one or more associated adverse symptoms or complications, or inhibition or reversal of one or more of the physiological, biochemical or cellular manifestations or characteristics of the condition. A therapeutic benefit or improvement (“ameliorate” is used synonymously) therefore need not be destruction of all target cells (e.g., tumor) or ablation of any particular symptom or complication or all adverse symptoms or complications associated with the disorder. For example, inhibiting an increase in tumor cell mass (stabilization of the tumor) can prolong lifespan even if only for a few days, weeks or months, even though some or the majority of the tumor remains. Stabilization may even provide benefits in reduction or stabilization of symptoms without an increase of life expectancy.

Specific non-limiting examples of therapeutic benefit include a reduction in tumor volume (size or cell mass), inhibiting an increase in tumor volume, slowing or inhibiting tumor progression or metastasis, stimulating tumor cell lysis, necrosis or apoptosis, and reducing tumor metastasis. Examination of a biopsied sample containing a tumor (e.g., blood or tissue sample), can establish whether a reduction in numbers of tumor cells or inhibition of tumor cell proliferation has occurred. Alternatively, for a solid tumor, invasive and non-invasive imaging methods can ascertain a reduction in tumor size, or can ascertain inhibiting an increase in tumor size.

Adverse symptoms and complications associated with tumor, neoplasia, and cancer that can be reduced or decreased include, for example, nausea, lack of appetite, lethargy, pain and discomfort. Thus, a reduction in the severity, duration or frequency of adverse symptoms, an improvement in the subjects subjective feeling, such as increased energy, appetite, and psychological well being, are all examples of therapeutic benefit.

The doses or “sufficient amount” for treatment to achieve a therapeutic benefit or improvement are effective to ameliorate one, several or all adverse symptoms or complications of the condition, to a measurable or detectable extent. Preventing or inhibiting a progression or worsening of the disorder, condition or adverse symptom, is also a satisfactory outcome. Thus, in the case of a cell-proliferative condition or disorder, the amount will be sufficient to provide a therapeutic benefit to the subject or to ameliorate the condition or symptom. The dose may be proportionally increased or reduced as indicated by the status of the disease being treated or a side effect of the treatment. This is also referred to herein as an “therapeutically effective amount.”

Doses also considered effective are those that result in reduction of the use of another therapeutic regimen or protocol. For example, a therapeutic benefit is achieved with an H2A histone polypeptide of the invention if its administration results in less chemotherapeutic drug, radiation or immunotherapy being required for tumor treatment.

Of course, as is typical for treatment protocols, some subjects will exhibit greater or less response to treatment. For example, appropriate amounts will depend upon the condition treated (e.g., the tumor type or stage of the tumor), the therapeutic effect desired, as well as the individual subject (e.g., the bioavailability within the subject, gender, age, etc.).

The H2A histone polypeptide of the inventions and nucleic acids can be administered in association with any other therapeutic regimen or treatment protocol. Other treatment protocols include drug treatment (chemotherapy), surgical ressection, hyperthermia, radiotherapy, and immunetherapy, as set forth herein and known in the art. The invention therefore provides methods in which the H2A polypeptides and nucleic acids of the invention are used in combination with any anti-cell proliferative therapeutic regimen or treatment protocol, such as those set forth herein or known in the art.

Radiotherapy includes internal or external delivery to a subject. For example, alpha, beta, gamma and X-rays can administered to the subject externally without the subject internalizing or otherwise physically contacting a radioisotope. Specific examples of X-ray dosages administered range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease.

The term “subject” refers to animals, typically mammalian animals, such as a non-human primate (gorillas, chimpanzees, orangutans, macaques, gibbons), a domestic animal (dogs and cats), a farm animal (horses, cows, goats, sheep, pigs), experimental animal (mouse, rat, rabbit, guinea pig) and humans. Subjects include disease model animals (e.g., such as mice and non-human primates) for testing in vivo efficacy of the H2A polypeptides and nucleic acids of the invention (e.g., a tumor animal model). Human subjects include adults, and children, for example, newborns and older children, between the ages of 1 and 5, 5 and 10 and 10 and 18 years.

Subjects include humans having or at risk of having a cell-proliferative disorder, such as subjects having a cell or tissue that expresses AgRM1, or subjects that have a family history of, are genetically predisposed to, or have been previously afflicted with a hyperproliferative disorder. Thus, subjects at risk for developing cancer can be identified with genetic screens for tumor associated genes, gene deletions or gene mutations. Subjects at risk for developing breast cancer lack BRCA1, for example. Subjects at risk for developing colon cancer have deleted or mutated tumor suppressor genes, such as adenomatous polyposis coli (APC), for example. Subjects include candidates for anti-tumor or immune system-enhancing therapy, or those that are undergoing or have undergone an anti cell-proliferative or immune system-enhancing therapy (e.g., subjects in remission).

Further provided are methods of screening for or identifying an inhibitor or stimulator of expression of a histone H2A polypeptide that specifically binds to a human monoclonal antibody produced by the cell line deposited as ATCC accession no. CRL-12142. In one embodiment, a method includes: contacting a cell that expresses or is capable of expressing histone H2A polypeptide that specifically binds to a human monoclonal antibody produced by the cell line deposited as ATCC accession no. CRL-12142 with a test compound; and detecting expression of said histone H2A polypeptide that specifically binds to a human monoclonal antibody produced by the cell line deposited as ATCC accession no. CRL-12142. A change in expression indicates that the test compound is an inhibitor or stimulator of expression of a histone H2A polypeptide that specifically binds to a human monoclonal antibody produced by the cell line deposited as ATCC accession no. CRL-12142. In various aspects, the contacting is in solution, in solid phase, in vivo or in vitro.

Additionally provided are methods of screening for or identifying a subject having, or at risk of having, a cell proliferative disorder (e.g., a cell proliferative disorder in a tissue selected from breast, colon, gut, lung, brain, skin or pancreas). In one embodiment, a method includes: analyzing for the expression of a histone H2A polypeptide of about 19 kDa that specifically binds to a human monoclonal antibody produced by the cell line deposited as ATCC accession no. CRL-12142. The presence of a histone H2A polypeptide of about 19 kDa that specifically binds to a human monoclonal antibody produced by the cell line deposited as ATCC accession no. CRL-12142 in the tissue identifies the subject as having or at risk of having a cell proliferative disorder.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

The invention illustratively described herein can suitably be practiced in the absence of any element or elements, limitation or limitations that are not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalent of the invention shown or portion thereof, but it is recognized that various modifications are possible within the scope of the invention claimed. Thus, it should be understood that although the present invention has been specifically disclosed by preferred embodiments and optional features, modifications and variations of the inventions embodied herein disclosed can be readily made by those skilled in the art, and that such modifications and variations are considered to be within the scope of the inventions disclosed herein. The inventions have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless or whether or not the material to be removed was specifically recited. In addition, where features or aspects of an invention are described in terms of the Markush group, those schooled in the art will recognize that the invention is also thereby described in terms of any individual member or subgroup of members of the Markush group. Further, when a reference to an aspect of the invention lists a range of individual members, as for example, ‘SEQ ID NO: I to SEQ ID NO: 100, inclusive,’ it is intended to be equivalent to listing every member of the list individually, and additionally it should be understood that every individual member may be excluded or included in the claim individually.

The steps depicted and/or used in methods herein may be performed in a different order than as depicted and/or stated. The steps are merely exemplary of the order these steps may occur. The steps may occur in any order that is desired such that it still performs the goals of the claimed invention.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims.

As used herein, singular forms “a”, “and,” and “the” include plural referents unless the context clearly indicates otherwise. Thus, for example, reference to “an H2A polypeptide” includes a plurality of H2A polypeptides, and reference to “a sequence” can include reference to all or a potion of or one or more sequences, and so forth.

Further, all patents and publications described herein are hereby incorporated by reference to the same extent as if each individual patent or publication was specifically and individually indicated to be incorporated by reference, and including any drawings figures or tables.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

AgRM1 was identified using an antibody that binds to the antigen in a Western Blot. The band was excised and the protein sequenced. The AgRM1 polypeptide is a truncated version of histone H2A (FIG. 1). The beginning amino terminal residue of AgRM1 was identified as residue #52 of normal H2A. The gene sequence was identified from mRNA isolated from the NCI-H661 cell line (available from the ATCC) and sequenced by standard methods. The sequences are illustrated in FIG. 1. Other forms of AgRM1 were shown to have the sequence AAVLEYLTAEILELA at or near the amino-terminus of the AgRM1 polypeptide.

### Example 2

Development of Murine Monoclonal Antibody

Monoclonal antibodies are developed by standard means known in the art. In one embodiment, AgRM1 is partially purified. Mice are immunized via standard techniques. Following the final immunization the mice are splenectomized. The sensitized splenocytes are mixed with murine myeloma cells such as SP2/0 cells (American Type Culture Collection, Rockville, Md.) and are fused, e.g., using polyethylene glycol 1500 as described by Oi and Herzenberg (Oi and Herzenberg, In Selective Matters in Cell Immunology, Ed. D. B. Mishell and S. M. Shiji, p.351-372, (1980)).

The fused cells are transferred to 96-well microculture plates and cultured for 2 weeks in selective media to obtain lymphocyte-myeloma hybrid cells.

The hybridomas are screened for the presence of antibody to the partially purified antigen preparation. Hybridomas that screen positive for the presence of antibody to the partially purified antigen preparation are cloned. The antibody subtype is determined by means known in the art.

### Example 3

The methods used for xenograft studies are as follows. On day zero, fifteen female athymic mice (nu/nu; 4-6 weeks old) were each injected with 5×10⁶(10e6)−1×10⁷(10e7) of the target cells (injections in the left flank) and divided into three groups of five mice each. On day 7, Group 1 received 100 μl (microliter) injections of PBS, Group 2 received 100 μl (microliter) injections of 100 μg (microgram) of control (unrelated) IgG, and Group 3 received 100 μl (microliter) injections of 100 μg (microgram) of the test RM antibody. On day 10, each Group received a second injection of their respective treatments. On day 14 each Group received a third injection and on day 21 each Group received their last injection. Each mouse was evaluated and tumor measured on the same day, once a week.

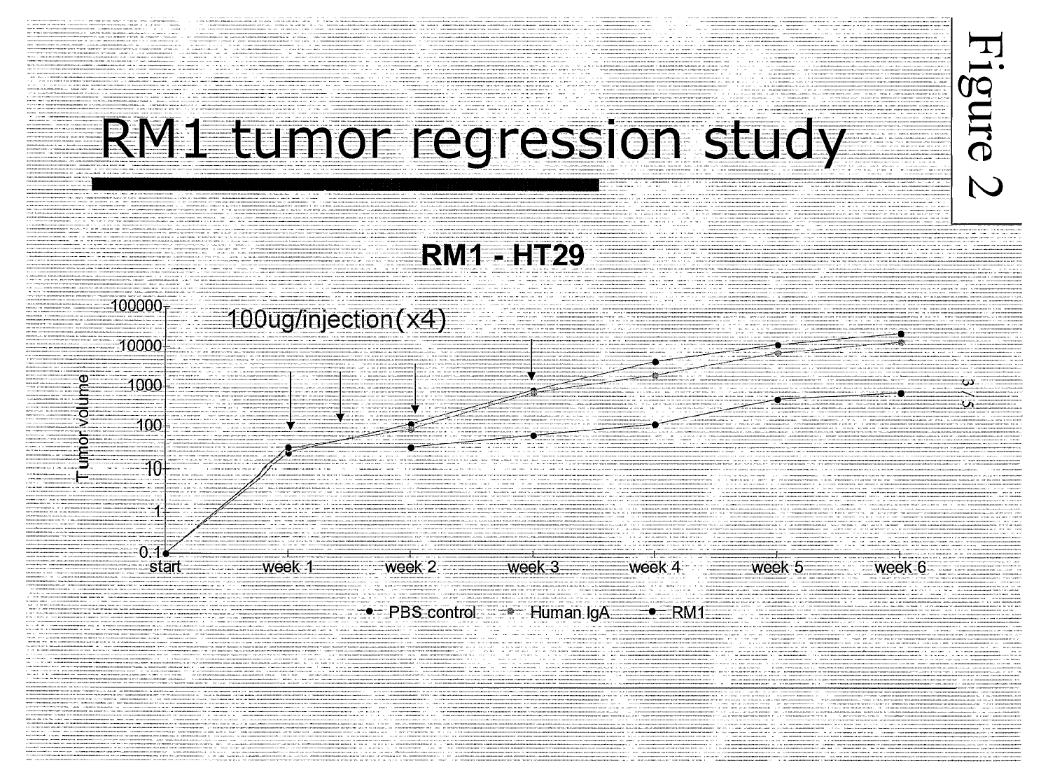

The results are shown in FIG. 2.

### Example 4

The methods used for xenograft studies are as follows. On day zero, twenty-five female athymic mice (nu/nu; 4-6 weeks old) were each injected with 5×10⁶(10e6) to 1×10⁷(10e7) of the target cells (injections in the left flank) and divided into five groups of five mice each. On day 7, Group 1 received 100 μl (microliter) injections of PBS, Group 2 received 100 μl (microliter) injections of 100 μg (microgram) of control (unrelated) IgG, and Group 3 received 100 μl (microliter) injections of 100 μg (microgram) of the test RM antibody, and Groups 3, 4, and 5 received 1001 (microliter) injections of 100 μg (microgram) of the respective test RM antibody. On day 10, each Group received a second injection of their respective treatments. On day 14 each Group received a third injection and on day 21 each Group received their last injection. Each mouse was evaluated and tumor measured on the same day, once a week.

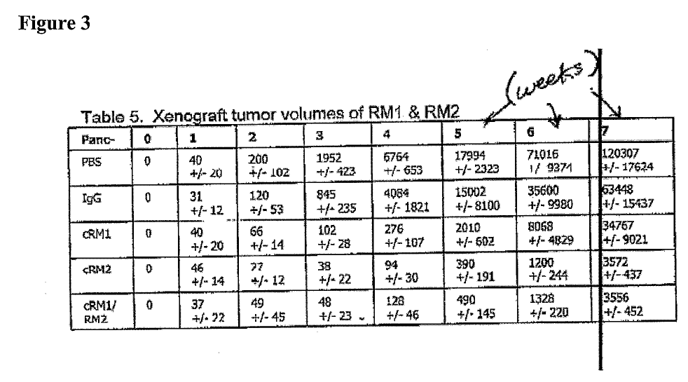

The results are shown in FIG. 3.

### Example 5

Cell lines are stained with the anti-RM1 antibody by standard methods of cell line staining: EIA and FACS.

EIA method. Logarithmic phase growing cells were collected, washed in PBS, resuspended, aliquoted at 2×10⁵(10e5) cells per well in flat-bottomed Immulon 96-well plates, and placed overnight in a 37° C. drying oven. To these cells, antibody supernatant was added, incubated, washed, and developed with horseradish peroxidase-conjugated goat anti-human IgG. All tests were done in triplicate and read on a micro-plate EIA reader.

FACS method. Target cells were harvested using EDTA while in mid-log phase of growth and 1×10⁵(10e5) cells were placed in each assay tube. 100 μl (microliter) of test antibody, at 5-10 ug/ml (microgram/ml), were added to each tube, incubated overnight at 4° C., washed 3× in RPMI 1640 plus 10% FCS. FITC-conjugated goat anti-human IgG (American Qualex) was added to each tube, incubated for 30 min, washed 3×, and read on an EIA reader.

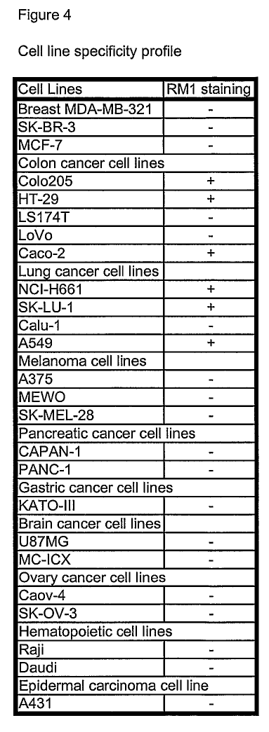

The results are shown in FIG. 4. The anti-RM1 antibody stains the cell membrane of the following cell lines: Colo2O5 cells, HT-29 cells, Caco-2 cells, NCI-H661 cells, SK-LU-1 cells, and A549 cells. Therefore the RM1 antigen is detected on the cell surface of colon cancer cell lines and lung cancer cell lines.

1. An isolated polypeptide comprising a carboxy-terminal portion of an H2A polypeptide sequence that specifically binds to a human monoclonal antibody produced by the cell line deposited as ATCC accession no. CRL-12142 wherein said isolated polypeptide comprising a carboxy-terminal portion of an H2A amino acid sequence has a deletion of at least 45 amino acids from its amino terminus relative to full length native human histone H2A.

2. The isolated polypeptide of claim 1 wherein said deletion from said amino terminus is between 45 and 55 amino acids from said amino terminus relative to full length native human histone H2A.

3. (canceled)

4. The isolated polypeptide of claim 1 wherein said deletion from said amino terminus is 51 amino acids from its amino terminus relative to full length native human histone H2A.

5. (canceled)

6. The polypeptide of claim 1, wherein said H2A polypeptide sequence is mammalian.

7. The polypeptide of claim 1, wherein said H2A polypeptide sequence is human.

8. The polypeptide of claims 1, wherein said H2A polypeptide sequence is an H2A variant.

9. The polypeptide of claim 8, wherein said H2A variant comprises a polypeptide encoded by a sequence selected from Genbank Accession Nos. AF058445; AF058446; AF044286; AF058444; NM138609; NM138609; BC013331; AF054174; AF041483; NM138610.1; and NM004893.2.

10. A nucleic acid consisting essentially of a DNA sequence encoding the polypeptide of claim 1.

11. A vector comprising a nucleic acid consisting essentially of a DNA sequence encoding the polypeptide of claim 1.

12. A transformed cell comprising a nucleic acid encoding said polypeptide of claim 1.

13. The transformed cell of claim 12, wherein said cell is prokaryotic.

14. The transformed cell of claim 12, wherein said cell is eukaryotic.

15. The transformed cell of claim 14, wherein said cell is a fungal cell, an insect cell or a mammalian cell.

16. A kit comprising said polypeptide of claim 1.

17. A pharmaceutical composition comprising said polypeptide of claim 1, and a pharmaceutically acceptable carrier.

18. A kit comprising the pharmaceutical composition of claim 17.

19. A method of inducing or increasing an immune response to a histone H2A polypeptide sequence that specifically binds to a human monoclonal antibody produced by a cell line deposited as ATCC accession no. CRL-12142, comprising administering to a patient a therapeutically effective amount of said histone H2A polypeptide sequence that specifically binds to said human monoclonal antibody produced by said cell line deposited as ATCC accession no. CRL-12142 to elicit an immune response to said histone H2A polypeptide sequence in said patient.

20. The method of claim 19 wherein said histone H2A polypeptide administered to said patient has an amino terminal deletion of at least 45 amino acids from its amino terminus relative to full length native human histone H2A.

21. The method of claim 19 wherein said deletion from said amino terminus is between 45 and 55 amino acids from its amino terminus relative to full length native human histone H2A.

22. (canceled)

23. The method of claim 19 wherein said deletion from said amino terminus is 51 amino acids from its amino terminus relative to full length native human histone H2A.

24. (canceled)

25. The method of claim 19, wherein said H2A polypeptide sequence is mammalian.

26. The method of claim 19, wherein said H2A polypeptide sequence is human.

27. The method of claim 19, wherein said H2A polypeptide sequence is an H2A variant.

28. The polypeptide of claim 27, wherein said H2A variant comprises a polypeptide encoded by a sequence selected from Genbank Accession Nos. AF058445; AF058446; AF044286; AF058444; NM138609; NM138609; BC013331; AF054174; AF041483; NM138610.1; and NM004893.2.

29. (canceled)

30. (canceled)

31. A method of treating a cell proliferative disorder, comprising administering to a patient a therapeutically effective amount of a histone H2A polypeptide sequence that specifically binds to a human monoclonal antibody produced by a cell line deposited as ATCC accession no. CRL-12142 to treat said cell proliferative disorder.

32. The method of claim 31, wherein said cell proliferative disorder comprises cells selected from breast, colon, gut, lung, brain, skin and pancreas.

33. A method of treating a patient having, or at risk of having a tumor, comprising administering to said patient a therapeutically effective amount of a histone H2A polypeptide sequence that specifically binds to a human monoclonal antibody produced by a cell line deposited as ATCC accession no. CRL-12142 to treat said patient.

34. (canceled)

35. The method of claim 33 wherein said deletion from said amino terminus is between 45 and 55 amino acids from its amino terminus relative to full length native human histone H2A.

36. (canceled)

37. The method of claim 33 wherein said deletion from said amino terminus is 51 amino acids from its amino terminus relative to full length native human histone H2A.

38. (canceled)

39. The method of claim 33, wherein said H2A polypeptide sequence is mammalian.

40. The method of claim 33, wherein said H2A polypeptide sequence is human.

41. The method of claim 33, wherein said H2A polypeptide sequence is an H2A variant.

42. The polypeptide of claim 41, wherein said H2A variant comprises a polypeptide encoded by a sequence selected from Genbank Accession Nos. AF058445; AF058446; AF044286; AF058444; NM138609; NM138609; BC013331; AF054174; AF041483; NM138610.1; and NM004893.2.

43. The method of claim 33, wherein said tumor is metastatic.

44. The method of claim 33, wherein said tumor comprises a stage I, II, III, IV or V tumor or cancer.

45. The method of claim 33, wherein said tumor comprises a solid tumor.

46. The method of claim 33, wherein said tumor comprises a sarcoma, carcinoma, melanoma, myeloma, blastoma, glioma, lymphoma or leukemia.

47. The method of claim 45, wherein said tumor comprises cells selected from breast, colon, gut, lung, brain, skin or pancreas.

48. The method of claim 45, wherein said tumor comprises cells selected from the lung.

49. The method of claim 45, wherein said tumor comprises cells selected from the colon.

50. The method of claim 33, wherein said patient is a candidate for, is undergoing, or has undergone an anti-tumor or immune system-enhancing therapy.

51. The method of claim 33, further comprising administering an anti-tumor or immune system-enhancing agent or treatment.

52. The method of claim 33, further comprising administering an antibody, radioisotope, radiation, a toxic, immunotherapeutic or chemotherapeutic agent, immunotherapy, surgical resection, or hyperthermia.

53. (canceled)

54. (canceled)

55. A method of identifying an inhibitor or stimulator of expression of a histone H2A polypeptide sequence that specifically binds to a human monoclonal antibody produced by a cell line deposited as ATCC accession no. CRL-12142, comprising:
a) contacting a cell that expresses or is capable of expressing histone H2A polypeptide sequence that specifically binds to a human monoclonal antibody produced by a cell line deposited as ATCC accession no. CRL-12142 with a test compound;
b) detecting expression of said histone H2A polypeptide sequence that specifically binds to a human monoclonal antibody produced by said cell line deposited as ATCC accession no. CRL-12142, wherein a change in expression indicates that said test compound is an inhibitor or stimulator of expression of said histone H2A polypeptide sequence that specifically binds to a human monoclonal antibody produced by said cell line deposited as ATCC accession no. CRL-12142; and
c) comparing said the inhibition or stimulation of binding to the inhibition of antibody binding in the presence of a carboxy-terminal fragment of a histone H2A.

56. The method of claim 55, wherein said contacting is in solution, in solid phase, in vivo or in vitro.

57. A method of screening a subject having, or at risk of having, a cell proliferative disorder, said cell proliferative disorder in a tissue selected from breast, colon, gut, lung, brain, skin or pancreas, comprising: analyzing for expression of a histone H2A polypeptide sequence of that migrates under conditions of denaturing electrophoresis at about 19 kDa, and that specifically binds to a human monoclonal antibody produced by the cell line deposited as ATCC accession no. CRL-2142, wherein the presence of said histone H2A polypeptide sequence of that migrates under conditions of denaturing electrophoresis at about 19 kDa, and that specifically binds to a human monoclonal antibody produced by the cell line deposited as ATCC accession no. CRL-12142 in the tissue identifies said subject as having, or at risk of having, a cell proliferative disorder.
